(19) United States
(12) Patent Application Publication
(10) Pub. No.: US 2019/0022365 A1
(43) Pub. Date: Jan. 24, 2019

(54) MICRONEEDLE DEVICE

(71) Applicant: NEMAURA PHARMA LIMITED, Loughborough (GB)

(72) Inventors: Dewan Fazlul Hoque CHOWDHURY, Loughborough (GB); Waqas Akbar LUGHMANI, Loughborough (GB)

(73) Assignee: Nemaura Pharma Limited, Loughborough (GB)

(21) Appl. No.: 16/072,875

(22) PCT Filed: Jan. 26, 2017
(86) PCT No.: PCT/GB2017/050198

§ 371 Date: Jul. 25, 2018

(30) Foreign Application Priority Data
Jan. 27, 2016 (GB) 1601486.2

(51) Int. Cl. A61M 37/00

(52) U.S. Cl. CPC A61M 37/0015; A61M 2037/0023; A61M 2037/003; A61M 2037/0046; A61M 2037/0061

(57) ABSTRACT

A microneedle device comprising: a substrate comprising one or more layers; and at least one microneedle which extends from the substrate; wherein the substrate comprises at least one layer which is a high surface energy material layer and which is capable of retaining the at least one microneedle. A method of manufacturing the microneedle device, a method of applying a microneedle assembly to the skin of a patient, and a method of delivering a therapeutic agent to a patient in need thereof using a microneedle are also provided

The invention relates generally to a microneedle device, and in particular to a microneedle device for which the needle has an improved mechanical fit with the substrate.

### BACKGROUND

Microneedle systems are commonly used for the delivery of therapeutic agents and other substances through the skin. In particular, microneedle devices are provided with a hollow microneedle array which is attached to a drug reservoir. The microneedles pierce the skin and remain attached while the drug flows from the reservoir into the skin.

Microneedle systems generally include two parts, which are identified as the cannula portion and the substrate portion. In order to pierce the skin, the cannula portion, i.e. the needle, must be constructed of a hard material, for example, a metal such as stainless steel. The substrate is typically manufactured more economically using polymers such as polypropylene and silicone.

The most economical arrangement is to provide a microneedle system including a cannula portion which is comprised of metal and a substrate which is comprised of a polymer. However, when these two substances are employed in combination, it is difficult to provide a suitable means for securing the parts together. The most suitable substance for securing the metal portion and the substrate portion together is typically an adhesive substance such as a cyanoacrylate or the like.

Conventional adhesive substances of this type have a good affinity for the metallic portion, or in other words, the adhesive substance will form a very good bond with the metal. On the other hand, this type of adhesive substance does not form a good bond with the material of the substrate. A particular problem then arises as to the manner in which a sufficiently good bonding of microneedle can be obtained with the substrate in order that it can pass the pull test which is ordinarily required for the needle to meet minimum standards required in the medical field. It is apparent that the microneedle must be fixed with respect to the substrate to a sufficient extent that the needle will not move when the needle is inserted into the skin or needle is withdrawn from a patient's skin.

This problem is compounded by the fact that the adjacent surfaces of the needle and the substrate are of very small dimensions thereby providing a very small area for obtaining the necessary holding power between the parts.

In a typical example, wherein the needle may have a diameter of no more than approximately 0.2 millimetre, and further wherein the length of the adjacent surfaces on the needle and the substrate are on the order of 0.6 millimetre in length, the total area available for obtaining a good interconnection between the needle and the substrate is only about 0.018 square millimetres. It is evident that this very small area does not provide adequate contact area for obtaining a very effective adhesive bond.

Thus, a need exists for a microneedle device in which the needle has an improved mechanical fit with the substrate. In particular, an object of the present invention is the provision of enough holding power of the microneedle during the pull-out test.

A further object of the present invention is to provide a disposable microneedle device which can be manufactured as economically as possible, and yet which is efficient and reliable in use.

According to the present invention, there is provided a microneedle device comprising:

a substrate comprising one or more layers; and

at least one microneedle which extends from the substrate;

wherein the substrate comprises at least one layer which is a high surface energy material layer and which is capable of retaining the at least one microneedle.

Preferably, the substrate further comprises an upper layer and a lower layer, and wherein the high surface energy material layer is sandwiched between the upper layer and the lower layer.

Conveniently, the upper layer and the lower layer can comprise of at least one of a polymer, plastic, metal, ceramic, glass or a combination of any of these.

Advantageously, the upper layer and/or lower layer comprises a methacrylic polymer, silicone, polyethylene terephthalate, polyethylene, polypropylene, polyester, or polyurethane, more preferably wherein the upper and/or lower layer comprises silicone.

Preferably, the microneedle is comprised of a hard material selected from plastic, metal, polymer and ceramic, preferably wherein the microneedle is comprised of a metal, more preferably wherein the microneedle is comprised of stainless steel.

Conveniently, the substrate further comprises at least one hole which extends through the substrate, and from which the at least one microneedle extends substantially perpendicularly from the substrate.

Advantageously, the microneedle is further affixed to the substrate with an adhesive.

Preferably, the adhesive is a medical grade adhesive which is self-curing or heat cured, preferably wherein the adhesive is an acrylate adhesive, more preferably wherein the adhesive is cyanoacrylate.

Conveniently, the high surface energy material layer is a plastic, metal, polymer, ceramic or a combination of one or more of these having a surface energy of from 250 to 1500 mJ/m².

Advantageously, the high surface energy material layer is comprised of stainless steel, preferably wherein the high surface energy material layer is a stainless steel mesh.

Preferably, the device is adapted to be used with a drug reservoir.

Conveniently, the layers of the substrate are held together using an adhesive.

Advantageously, the needle is retained mounted in the substrate with a needle height area of contact of 1 mm or less.

Preferably, there is a gap between the needle and the substrate which is filled with adhesive and allows some movement or flow of the adhesive upon pulling of the needle.

According to the present invention, there is provided a method of manufacturing the microneedle device according to the invention, wherein the method comprises:

(i) providing the one or more layers of the substrate;

(ii) providing the at least one microneedle; and

(iii) mounting the at least one microneedle in the substrate.

Preferably, the microneedle is further affixed to the substrate using an adhesive and the one or more layers are bonded together using an adhesive.

According to the present invention, there is provided a method of applying a microneedle device assembly according to the invention to the skin of a patient, wherein the method comprises the step of applying the microneedle device to the skin of the patient so that the at least one microneedle penetrates the surface of the skin.

According to the invention, there is provided a method of delivering a therapeutic agent to a patient in need thereof using a microneedle device of the invention, wherein the method comprises the step of applying the microneedle device to the skin of the patient so that the at least one microneedle penetrates the surface of the skin.

The present invention will now be described, by way of example, in which:

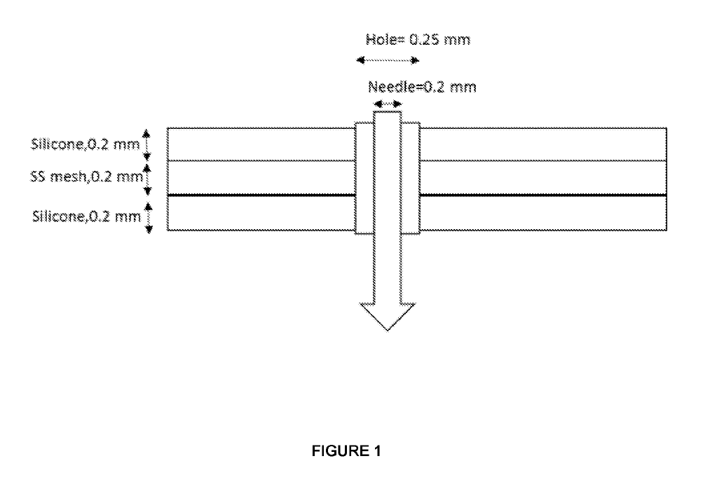

FIG. 1 is a cross section schematic of a microneedle device in accordance with the present invention showing the sandwich mesh and needle bonding.

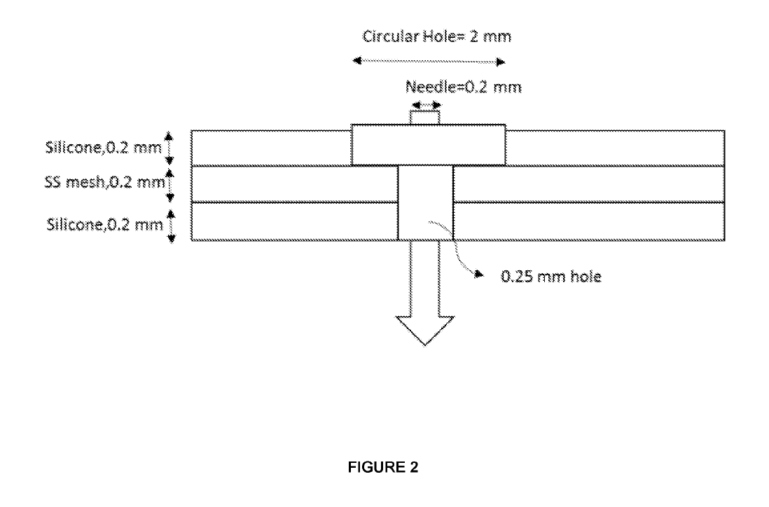

FIG. 2 is a cross section schematic of a microneedle device in accordance with the present invention showing a modified sandwich assembly.

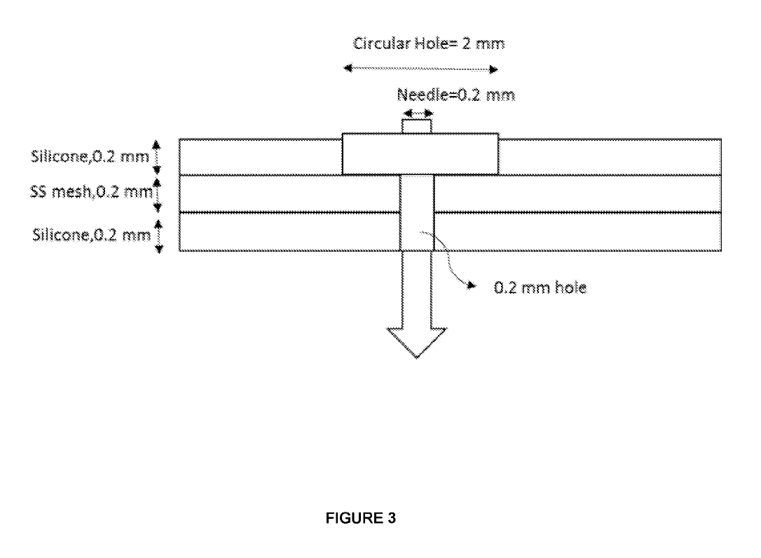

FIG. 3 is a cross section schematic of a microneedle device in accordance with the present invention showing a Press Fit with a modified sandwich.

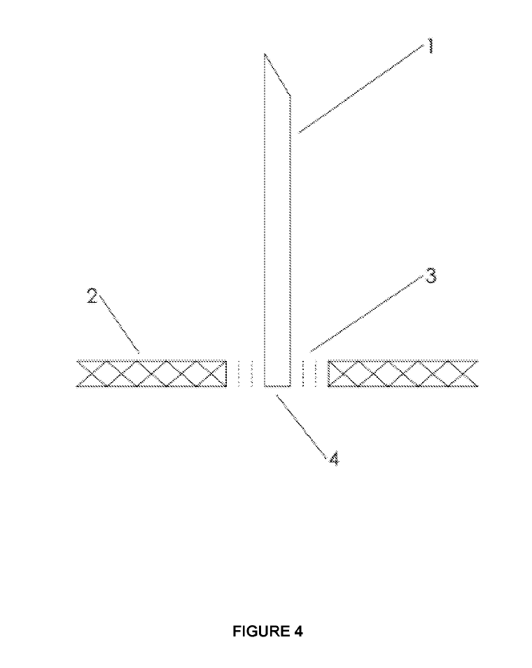

FIG. 4 is a cross section schematic of a needle shown in a vertical position with needle tip facing up adhered using adhesive to a mesh layer.

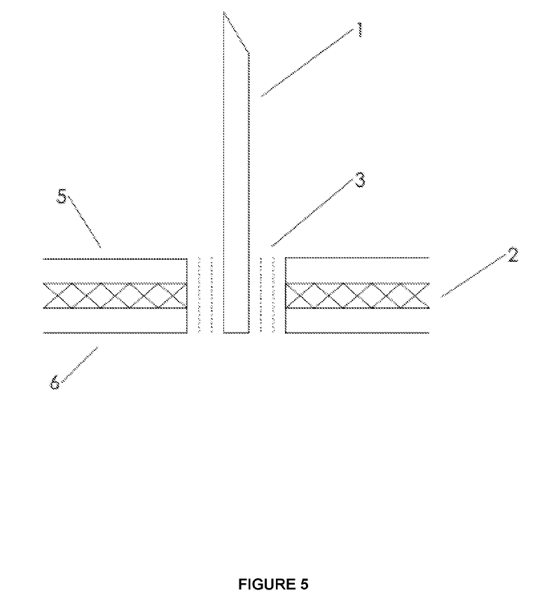

FIG. 5 is a cross section schematic of a needle in contact with adhesive, whereby the adhesive is bonded directly to the needle on one surface and a mesh and additional surface on the other surface of the needle.

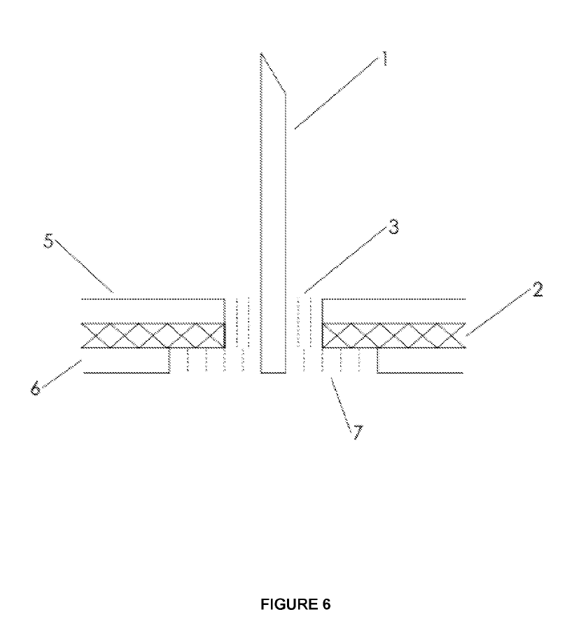

FIG. 6 is a cross section schematic representation of FIG. 5 with an increased distance between the substrate at the lower portion of the liquid exit portion of the needle.

### DESCRIPTION OF INVENTION

In accordance with the present invention there is provided a microneedle device for the delivery of therapeutic agents to the skin.

The microneedle device comprises at least one microneedle. In accordance with one embodiment, an array of microneedles is provided. The one or more microneedles may be hollow and are provided with a central bore which provides a channel through the skin for delivery of a therapeutic agent or drug from a drug reservoir into the body of a patient.

The device of the invention is preferably in the form of a patch. More preferably, the device of the invention is a microneedle patch which is in fluid communication with a drug reservoir.

Alternatively, the one or more microneedles may be simply coated with the therapeutic agent or drug prior to use so that the drug is carried on the surface of the needle when the skin is pierced.

The microneedles may be solid or hollow, and generally have either a sharp or blunt tip, but with an aspect ratio that allows skin penetration. The microneedles typically have a diameter in the range of from 10 to several hundred microns. The microneedles may exist as one or more microneedles. If there are a plurality of microneedles, then the needles may be arranged as an array. The arrays may be assembled in rows and the needle heights may be uniform or different depending on the surface onto which the microneedle device is to be applied to. Further details of suitable microneedles and the method of preparation is disclosed in WO2015/025139 and GB 2478363.

In order for the needle to able to push through human skin; the needle must be constructed of a hard material. The material must be structurally stable to avoid fracturing and buckling; and have sharp tips to facilitate puncturing. Preferably the material is stainless steel. However, other types of materials can be used, including but not limited to, plastics, metals, polymers and ceramics. The material which is selected for the needle typically has a high surface energy.

Surface energies of common substances are set out below.

TABLE 1
Surface energy chart

| Solid Surface | Energy (mJ/m²) |
|---|---|
| Copper | 1103 |
| Silver | 890 |
| Aluminium | 840 |
| Zinc | 753 |
| Tin | 526 |
| Lead | 458 |
| Stainless steel | 700-1100 |
| Glass porcelain | 250-500 |
| PVA | 37 |
| Polystyrene | 36 |
| Polyethylene | 31 |
| Polypropylene | 29 |
| Silicone | 20-25 |

Surface contact is fundamental to adhesive performance. Adhesion is the molecular force of attraction between dissimilar materials. The strength of attraction is determined by the surface energy of the material. The higher the surface energy, the greater the molecular attraction. The lower the surface energy, the weaker the attractive forces.

The microneedle device according to the present invention also comprises a substrate portion. The at least one microneedle is affixed to the substrate. The substrate portion is preferably comprised of one or more layers. The substrate portion can be manufactured of any suitable material, and preferably a flexible materials. The microneedle device according to the present invention is preferably disposable, therefore it is advantageous from an economic view point that the microneedle device components can be sourced cheaply. This is an advantage over existing MEMS-based liquid drug delivery systems which are expensive to produce.

Another important factor to consider is that the microneedle will be comprised of a high surface energy material. When a high surface energy material such as stainless steel is employed in combination with a low surface energy material (such as the polymers listed in Table 1), then there will be poor adhesion between these two substrates.

In existing microneedle devices, an adhesive substance (for example, cyanoacrylate) is used to bond the microneedles to the substrate. However, conventional adhesives typically form a good adhesive bond with the metal but a poor adhesive bond with a low surface energy material substrate such as silicone.

In order to increase the surface energy at the interface between the needle wall and the substrate, and also between the needle, adhesive and substrate, the Applicants have found that by providing a substrate which has at least one layer which is made from a high surface energy material layer, this can provide or assist in creating a contact between low surface energy materials and the microneedle.

In one embodiment, the substrate comprises one or more layers, preferably the substrate comprises two layers, more preferably the substrate comprises three or more layers. The thickness of each layer of the substrate is between about 0.05 mm and 1.0 mm.

In an embodiment, the substrate comprises at least one layer which is a high surface energy material layer. Preferably, wherein the high surface energy material layer is a plastic, metal, polymer, ceramic or a combination of one or more of these. The high surface energy material layer provides an increased bond between the needle and the substrate material. The surface energy of the high surface energy material layer is from about 250 to 1500 mJ/m², such as from about 650 to about 1250 mJ/m², and preferably from about 800 to 1100 mJ/m².

Preferably, wherein the high surface energy material layer is a mesh.

Optionally, wherein the high surface energy material is stainless steel, preferably wherein the stainless steel is the in the form of a mesh.

In an embodiment, the substrate comprises at least three layers. Wherein the substrate is formed at least of an upper layer, a lower layer, and a high surface energy material layer which is sandwiched between the upper layer and the lower layer.

Optionally, wherein the upper layer and the lower layer are comprised of at least one of a polymer, plastic, metal, ceramic, glass or a combination of any of these. Preferably, the substrate material is comprised of polymers, including but limited to, polypropylene, PVC, polyethylene and silicone. The skilled person in the art would understand the wide choice of substrate materials available to him.

Typically, the upper layer and lower layer may comprise of a material which is a lower surface energy material. The surface energy of the upper and/or lower layers may range from about 5 to 200 mJ/m², such as from about 10 to about 100 mJ/m² and preferably between about 20 to 25 mJ/m².

Preferably, the upper and/or lower layers are comprised of silicone or other flexible polymeric material of sufficient elasticity and plasticity, such as the methacrylic polymers, and other polymers that would be generally deemed impermeable to drugs, such as polyethylene terephthalate (PET), polyethylene, polypropylene, polyester, and polyurethane amongst others, which offer the desired flexibility.

Further additional layers are also possible, and will depend upon the application for which the microneedle device is used for. The function of these layers may be cosmetic, or may be to impede drug flow or diffusion into the adhesive regions where direct contact may lead to accelerated degradation of the drug for example, or cause the adhesive to become brittle. The above listed polymers or combinations thereof, and possibly in conjunction with plasticisers, and metallisation of the surface of the layers, may be suited for these additional layers.

In an embodiment, the microneedle is further affixed to the substrate using an adhesive. Where there are multiple substrate layers, they may also be held together using an adhesive. Preferably, the adhesive is a medical grade adhesive which is self-curing or heat cured. Preferably, the adhesive is an acrylate adhesive, and more preferably wherein the adhesive is cyanoacrylate.

Many types of adhesive are used in the medical field and would be suitable for use in the present invention. Polymeric-based adhesives are often classified as thermosets, thermoplastics, and elastomers. A thermosetting adhesive sets or cures into a network, normally through the action of heat, radiation or a catalyst. Some adhesives are derived from natural or organic materials, such as proteins, cellulose, or starch.

A preferred class of adhesives for use in the invention are acrylic materials, including cyanoacrylates, anaerobics, and modified acrylics. These adhesives are generally two-component systems, available in a range of different forms ready for mixing. Acrylic-based adhesives may be polymerized or cured using moisture, catalysts, heat, UV or visible light, or other sources of radiation. The polymerization of cyanoacrylates is initiated by the presence of moisture or of weak bases in the atmosphere or on the substrate.

Epoxy resins are another type of thermoset adhesives based on the epoxide group, which reacts to form the polymeric structure. Other suitable types of adhesive include polyurethanes, which are often based on polyethers or polyesters with terminating hydroxyl functional groups. The materials used in most polyurethane systems usually consist of one of several different formulations: di- or polyfunctional alcohols; polyhydroxy compounds (known as polyols); di- or polyfunctional isocyanates; or low-molecular-weight alcohols or amines.

Silicones (otherwise known as organopolysiloxanes) can also be used as an adhesive. The properties of the silicone depending on the nature of side groups and the interchain cross-linking. Curing can be performed catalytically, by heat or exposure to moisture.

Another class of adhesives are pressure-sensitive adhesives, based on elastomers (e.g., natural or synthetic rubbers), acrylics or hot-melt thermoplastics, tackifiers, or antioxidants.

As is known in the art, it can be important to ensure that the surfaces to be adhered by an adhesive are clean and suitable for the formation of a bond. The skilled person is also aware that materials need to be prepared, mixed, dispensed or applied, and cured where appropriate in order to give a suitable final result.

Preferably, wherein the substrate comprises one or more holes, wherein each of the one or more holes is configured to receive at least one microneedle. Preferably, wherein the at least one microneedle extends substantially perpendicularly from the hole in the substrate. The microneedles may be perpendicular to the surface or near perpendicular. Each of the holes has a diameter from about 0.04 mm to 1.2 mm, such as from about 0.1 to about 0.3 mm, and preferably is about 0.25 mm. In some embodiments, the hole within the substrate has a larger diameter in the upper layer of the substrate than the diameter of the hole when it extends through either of the lower layer and the sandwich.

Referring now particularly to FIGS. 4 to 6 inclusive, the invention is illustrated wherein the microneedle is indicated generally by reference numeral 1, and the substrate assembly is indicated generally by reference numeral 2. The microneedle may be formed of a suitable material such as stainless steel, and the substrate may be formed of silicone and the like. The micro needle includes a substantially cylindrical body inner diameter 8 and outer diameter 9, though it will be appreciated that microneedles can be fabricated to any desired shape using chemical etching, laser, micromachining and micro-molding techniques.

Substrate 2 is provided with three layers indicated by reference numerals 3, 4, 5, as seen in FIGS. 4 and 5. Layer 4 is a sandwich layer which may be formed of high surface energy material such as stainless steel. Substrate layers 3 and 5 are formed of low surface energy material, for example, silicone-like materials. Substrate layers can be combined together by any appropriate adhesive as discussed above.

Substrate layer 5 has a bore 6 completely therethrough and provides a base of adhesive meniscus which enhances the bonding strength. The bore 6 provides an opening for needle placement and also provides a gap for the adhesive.

In FIG. 4, cross section schematics of needle 1 are shown in the vertical position with the needle tip facing up, and adhered using adhesive 3 to mesh 4. The needle underside 4 (the side via which liquid would enter needle from a reservoir) is shown at the bottom of the substrate.

The mesh and the needle may be constructed from metal, plastics/polymers, ceramic or other material that provides an adequate surface energy on contact to provide the highest adhesive bonding force between each of these substrates and the adhesive. The adhesive is preferably a medical grade adhesive, and one that is self-curing, or cured by heat, time lapse, the application of physical pressure, sonic energy, or light energy.

FIG. 5 shows a cross section schematic of the needle 1 in contact with adhesive 3, whereby the adhesive is bonded directly to the needle on one surface and a mesh 2 and additional substrate 5, 6 on the other surface. The substrate 5 and 6 may be the same substrate or they may be different materials. The materials may be composed of flexible polymers, rigid polymers, or plastics, metal foil or sheets, or ceramic or glass. The key characteristic of these substrates is to ensure an enhanced bonding force between the adhesive and the substrate, thus increasing the pull force required to remove the needle from the adhesive.

The adhesive 3 is within a gap that allows some movement or flow of the adhesive on pulling of the needle, whereby the adhesive is one that is not brittle and has some tensile and flexural properties on curing and drying, to spread and pulling force over the adhesive first, before the needle is pulled from the substrate/adhesive; equally where the gap is minimal, the adhesive may provide the increased pull force by enabling the needle to be pulled in a direction perpendicular to the substrate whereby the adhesive has sufficient elasticity to flow sufficiently to enable the needle to move vertically for a greater distance than would be the case in the absence of the adhesive or in the presence of a non-flowing/non-elastic brittle adhesive.

FIG. 6 provides a cross section schematic representation of FIG. 5 with an increased distance between the substrate 6 at the lower portion of the device/liquid exit portion of the needle 1, whereby the increased distance is filled with further adhesive 7, with the benefit that it increases further the initial strain or load distribution that would occur when a pull force is applied to the needle tip (for example when it is inserted into and removed from the skin). It will be appreciated that whilst this illustrates a single recess with additional adhesive there may be further step-wise recesses to further increase the surface area of contact between substrate(s) and adhesive.

It is preferable that there is a membrane/substrate 5 present to occlude the adhesive that will draw into the mesh 2 from the adhesive 3 region. This substrate 5 may also have a membrane to cover adhesive 3 whereby substrate 5 is in direct contact around the circumference region of needle 1, to prevent any direct contact of the needle 1 with adhesive 3 to prevent any drug adsorbing or absorbing in to the adhesive and/or any residual matter leaching in to the skin. Substrate 6 may also similarly have a membrane extended to cover the entire adhesive 7 region, leaving just the needle 1 exit port exposed.

In general, needles are usually adhered to a plastic carrier for the purpose of attachment to a syringe, whereby the length of the needle is of the order of 10 mm in length in order to provide a significantly larger surface area of contact between needle wall and the plastic housing/carrier. In the present invention, however, we are adhering needles in a way that allows 2-3 mm or less height of needles to penetrate the skin—whilst the needle is retained mounted on a substrate with a needle height area of contact of 1 mm or less; this is technically very challenging and can be achieved using a mesh and polymer combination in order to increase the surface energy at the interface between the needle wall and the adhesive, and the adhesive and the mesh and polymer.

### Example

The follow ng samples were tested to establish pull-out force and bonding strength of a microneedle device prepared according to the present invention.

The sample size for the pullout experiments for each configuration was calculated using following equation.

$$n = {( \frac{\overline{σ} {z}_{α / 2}}{{e}_{r}} )}^{2}$$

where n is number of sample, eᵣ is the margin of error, z_{α/2} is the critical value of the standard normal distribution (found in tables of standard normal distribution) and σ̅ is the standard deviation of data.

The standard deviation of the experimental set up was calculated by pulling out needle from silicone substrate at the following conditions;

Sampling rate=1000 Hz.

Pullout rate=50 mm/min

The mean and standard deviations of the recorded averaged pullout force was calculated as:

X̅=0.57, σ̅=0.05, z_{α/2}=1.96 (at 95% level of confidence)

The margin of error acceptance was 0.05 N, and then the minimum number of sample required for testing was calculated as:

n=4

The following 5 configurations A-E were prepared and the pull-out force measured. The results are given below.

A. Stainless steel (SS) needle-SS substrate (Length×Diameter=0.2 mm×0.2 mm)

B. SS needle-Silicone (membrane, (not to be confused with silicone pressure sensitive adhesive) 0.2 mm×1 mm)

C. SS needle-Silicone-Sandwich SS mesh (0.6 mm×0.2 mm), 0.25 mm adhesive hole

D. SS needle-Silicone-Sandwich SS mesh (0.6 mm×0.2 mm), 2 mm Meniscus base hole

E. SS needle-Silicone-Sandwich SS mesh (0.6 mm×0.2 mm), 2 mm Meniscus base hole, 0.21 mm adhesive hole

TABLE 2
Surface Energy Table (reference)

| Material | Surface Energy (mJ/m²) |
|---|---|
| Stainless steel | 700-1100 |
| Silicone | 20-25 |

TABLE 3
Pullout force

| Embodiments | Maximum Pullout Force (N) |
|---|---|
| A | 8-10 N |
| B | 0.5-1 N |
| C | 2-2.5 N |
| D | 5-7 N |
| E | 6-8 N |

A. Stainless Steel Mesh and Needle Bonding

Mesh thickness=0.2 mm

Needle outer diameter=0.2 mm

Hole size in mesh for needle=0.25 mm

Adhesive=Cyanoacrylate (Apollo 2077), (Apollo 2028)

Predicted Force=joint length×shear strength of adhesive=2.4 N

Pullout rate=50 mm/min (Measured using ISO/DIS 7864)

| Sample | 1 | 2 | 3 | 4 | 5 | 6 | Average |
|---|---|---|---|---|---|---|---|
| Maximum | 10.8 | 6.2 | 9.3 | 10.6 | 6.8 | 8.7 | 8.6 |
| Force (N) | | | | | | | |

The reason for getting a higher than predicted value of force is the dispersion of adhesive along the mesh surface and meniscus formed by the adhesive around the needle. The results show that with the smaller dispersion area, the pull-out force is lower.

B. Needle and Silicone Bonding

Sample

| | 1 | 2 | 3 | 4 | Average |
|---|---|---|---|---|---|
| Maximum Force (N) | 0.5 | 0.63 | 0.45 | 0.54 | 0.53 |

C. Sandwich Mesh and Needle Bonding:

For the same adhesive and hole size as given in A. above.

| Sample | 1 | 2 | 3 | 4 | 5 | 6 | Average |
|---|---|---|---|---|---|---|---|
| Maximum | 2.2 | 2.45 | 2.3 | 2 | 2.1 | 1.95 | 2.1 |
| Force (N) | | | | | | | |

Without any dispersion of adhesive along mesh and meniscus formed with silicone substrate, we are getting approximately the same force as predicted by the formula.

D. Modified Sandwich Assembly:

| | Sample | | | | |
|---|---|---|---|---|---|
| | 1 | 2 | 3 | 4 | Average |
| Max. Force (N) | 7.0348 | 6 | 6.3 | 4.63 | 6 |

E. Press Fit with Modified Sandwich:

| Sample | 1 | 2 | 3 | 4 | 5 | 6 | Average |
|---|---|---|---|---|---|---|---|
| Maximum | 8.19 | 8.63 | 8.55 | 9.1 | 7.95 | 9.21 | 8.6 |
| Force (N) | | | | | | | |

1. A microneedle device comprising:
a substrate comprising one or more layers; and
at least one microneedle which extends from the substrate;
wherein the substrate comprises at least one layer which is a high surface energy material layer and which is capable of retaining the at least one microneedle.

2. The microneedle device according to claim 1, wherein the substrate further comprises an upper layer and a lower layer, and wherein the high surface energy material layer is sandwiched between the upper layer and the lower layer.

3. The microneedle device according to claim 2, wherein the upper layer and the lower layer can comprise of at least one of a polymer, plastic, metal, ceramic, glass or a combination of any of these.

4. The microneedle device assembly according to claim 3, wherein the upper layer and/or lower layer comprises at least one of a methacrylic polymer, silicone, polyethylene terephthalate, polyethylene, polypropylene, polyester, or polyurethane.

5. The microneedle device according to claim 1, wherein the microneedle comprises of a hard material selected from plastic, metal, polymer and ceramic.

6. The microneedle device according to claim 1, wherein the substrate further comprises at least one hole which extends through the substrate, and from which the at least one microneedle extends substantially perpendicularly from the substrate.

7. The microneedle device according to claim 1, wherein the microneedle is further affixed to the substrate with an adhesive.

8. The microneedle device according to claim 7, wherein the adhesive is a medical grade adhesive which is self-curing or heat cured.

9. The microneedle device according to claim 1, wherein the high surface energy material layer is a plastic, metal, polymer, ceramic or a combination of one or more of these having a surface energy of from 250 to 1500 mJ/m².

10. The microneedle device according to claim 9, wherein the high surface energy material layer comprises stainless steel.

11. The microneedle device according to claim 1, wherein the device is adapted to be used with a drug reservoir.

12. The microneedle device according to claim 2, wherein the layers of the substrate are held together using an adhesive.

13. The microneedle device according to claim 1, wherein the needle is retained mounted in the substrate with a needle height area of contact of 1 mm or less.

14. The microneedle device according to claim 1, wherein there is a gap between the needle and the substrate which is filled with adhesive and allows some movement or flow of the adhesive upon pulling of the needle.

15. A method of manufacturing the microneedle device according to claim 1, wherein the method comprises:
providing the one or more layers of the substrate;
providing the at least one microneedle; and
mounting the at least one microneedle in the substrate.

16. The method according to claim 15, wherein the microneedle is further affixed to the substrate using an adhesive and the one or more layers are bonded together using an adhesive.

17. A method of applying a microneedle device assembly according to claim 1 to the skin of a patient, wherein the method comprises the step of applying the microneedle device to the skin of the patient so that the at least one microneedle penetrates the surface of the skin.

18. A method of delivering a therapeutic agent to a patient in need thereof using a microneedle device according to claim 11, wherein the method comprises the step of applying the microneedle device to the skin of the patient so that the at least one microneedle penetrates the surface of the skin.
